(19) United States
(12) Patent Application Publication
(10) Pub. No.: US 2022/0117781 A1
(43) Pub. Date: Apr. 21, 2022

(54) WIRELESS INJECTOR

(71) Applicant: Alcon Inc., Fribourg (CH)

(72) Inventor: Paul R. Hallen, Colleyville, TX (US)

(21) Appl. No.: 17/494,283

(22) Filed: Oct. 5, 2021

Related U.S. Application Data
(60) Provisional application No. 63/092,048, filed on Oct. 15, 2020.

(51) Int. Cl. A61F 9/00; A61M 5/172

(52) U.S. Cl. CPC A61F 9/0017; A61M 2205/50; A61M 2210/0612; A61M 5/172

(57) ABSTRACT

The present disclosure generally relates to devices and methods for intraocular fluid delivery. Embodiments described herein provide improved mechanisms for precise delivery of therapeutic agents to intraocular tissues by utilizing a foot controller to wirelessly control a handheld injection device. The utilization of a remote foot controller to control the injection reduces or eliminates uneven application of injection force and hand tremor caused by hand-triggered devices, thus enabling precise position and flow rate control and reducing the risk of tissue damage.

### PRIORITY CLAIM

This application claims the benefit of priority of U.S. Provisional Patent Application Ser. No. 63/092,048 titled “WIRELESS INJECTOR,” filed on Oct. 15, 2020, whose inventor is Paul R. Hallen, which is hereby incorporated by reference in its entirety as though fully and completely set forth herein.

### BACKGROUND

### Field

Embodiments of the present disclosure generally relate to methods and devices for ophthalmic procedures, and more particularly, to methods and devices for intraocular fluid delivery.

### Description of the Related Art

Successful treatment of eye diseases and disorders depends not only on the effectiveness of therapeutic agents, but also on the effective administration thereof. Currently, the three primary methods of delivering therapeutic agents to the eye include systematic, topical, and intraocular administration. Compared to systematic and topical methods, intraocular administration offers the benefits of direct delivery of therapeutic agents and other fluids to target intraocular tissues at desired concentrations. Thus, intraocular drug delivery is frequently used in the treatment of many vitreoretinal diseases, including age-related macular degeneration (AMD), diabetic macular edema (DME), proliferative diabetic retinopathy, and retinopathy of prematurity (ROP), among others.

Typically, intraocular drug delivery requires controlled dispensing while maintaining precise position control in order to deliver a precise volume of fluid to a precise location within the eye without causing damage thereto. Controlled dispensation of the drug while maintaining precise position control may also be important when delivering expensive therapeutic agents, such as retinal gene therapies, so that as little of the therapeutic agent as possible is delivered off-target and wasted. However, conventional hand-operated injection devices present a number of challenges to a user (e.g., physician) when delivering fluids to intraocular tissues, which can result in imprecise drug delivery and/or damage to ocular tissues.

Injection devices typically include a syringe and a needle and fall into one of two categories—manual injection devices and automatic injection devices. With a manual injection device, a user must provide the mechanical force to drive the fluid through the device and into the eye, such as by pressing against a plunger during the injection. Typically, the user utilizes the same hand to control the position of the injection device and the flow rate of the fluid therethrough. As a result, the user may not be able to precisely control the flow rate or amount of injection, particularly if injection forces are too high for the user and/or if the plunger is extended too far. The combination of injection forces and extension of the plunger may cause shaking of the user's hand, which in turn may result in imprecise drug delivery and/or damage to ocular tissues.

Automatic injection devices overcome some of the challenges presented by manual injection devices by providing an automated mechanism to drive the fluid through the device. However, conventional automatic injection devices require hand-operated triggering by the user in order to activate the automated fluid-driving mechanism, which may cause undesired jerking of the device. During intraocular drug delivery, the uneven forces and tremors from the user's hand when activating the fluid-driving mechanism may be magnified in the eye and cause damage thereto, and further reduce injection control.

Accordingly, what is needed in the art are improved methods and devices for intraocular fluid delivery.

### SUMMARY

The present disclosure generally relates to methods and devices for intraocular fluid delivery.

In one embodiment, a handheld fluid injection device includes a handpiece having an interior compartment and a distal port configured to receive and engage a syringe, a plunger movably disposed within the interior compartment and having a distal end configured to slidably engage with a cavity of the syringe, and a drive unit operatively coupled to the plunger. The drive unit further includes a wireless communication module that is in wireless communication with an input device that enables the drive unit to control operations of the plunger based on wireless communication received from the input device for injection of fluids from the syringe.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1 illustrates a perspective view of an exemplary foot controller according to certain embodiments of the present disclosure.

FIG. 2 illustrates a perspective view of an exemplary surgical console according to certain embodiments of the present disclosure.

FIG. 3 illustrates a cross-sectional side view of a wireless automatic injector according to certain embodiments of the present disclosure.

FIG. 4 illustrates a cross-sectional side view of a wireless automatic injector according to certain embodiments of the present disclosure.

FIG. 5 illustrates a functional diagram of a wireless automatic injector wireless coupled to a foot controller and surgical console according to certain embodiments of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present disclosure generally relates to devices for intraocular fluid delivery. As just one example, the instruments described herein may be used for sub-retinal injection of therapeutic agents, such as gene therapies for ocular disease. However, the instruments described herein may be used in connection with any other intraocular fluid deliveries, as one of ordinary skill in the art appreciates.

Intraocular drug delivery may be used for the treatment of vitreoretinal disease due to the benefit of direct delivery into the vitreous, retina, and other ocular tissues. However, hand-delivered intraocular injections require great skill and precision due to the size and structure of the eye, and can become problematic from application of uneven forces or tremors from a surgeon's hands, which may result in damage to the patient's eye. Adverse events may also arise from a surgeon not being able to precisely control the flow rate or amount of fluid being injected through a hand-operated device, thus creating further delays and difficulties during ophthalmic procedures. The devices and methods described herein provide improved mechanisms for precise delivery of therapeutic agents to intraocular tissues by utilizing a foot controller to wirelessly control a handheld injection device. The utilization of a remote foot controller to control the injection reduces or eliminates uneven application of injection force and hand tremor caused by hand-triggered devices, thus enabling precise position and flow rate control and reducing the risk of tissue damage.

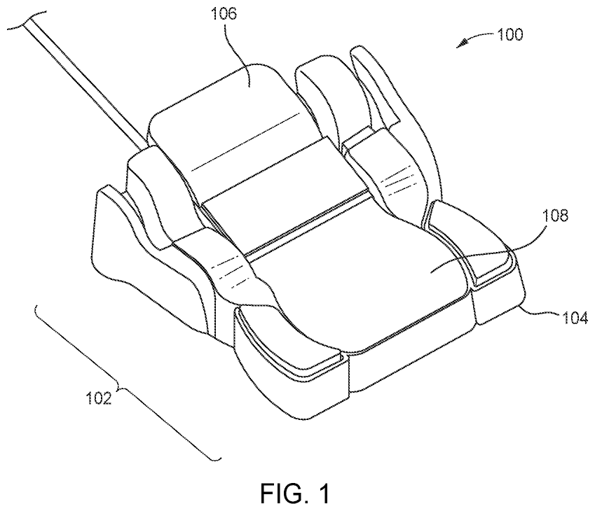

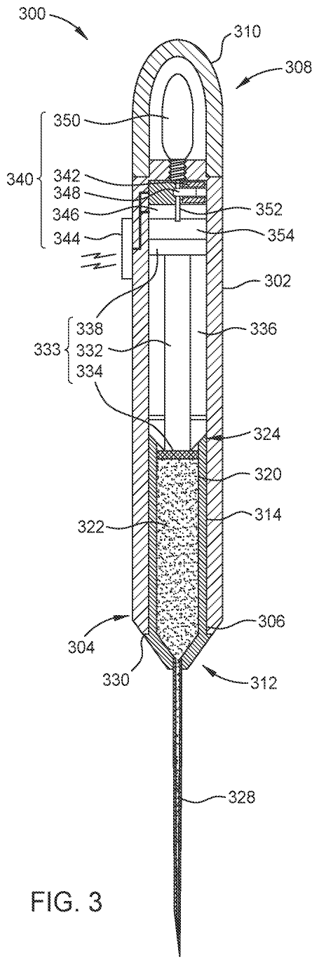

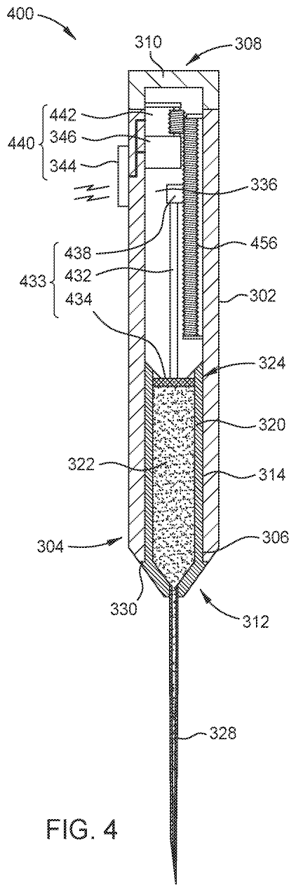

FIG. 1 illustrates a perspective view of an exemplary foot controller 100, in accordance with certain embodiments of the present disclosure. The foot controller 100 includes a body 102 with a base 104 that supports the foot controller 100 on an operating room floor. The body 102 further includes a footpedal 106, which is configured to be actuated by a user to perform one or more actions of a surgical procedure, such as injecting fluid from a handheld injection device (e.g., shown in FIGS. 3 and 4). For example, a surgeon depresses the footpedal 106 using the distal portion of his or her foot to move from a fully undepressed position to, for example, a fully depressed position in which the footpedal 106 lies in generally the same plane as a heel rest 108. Accordingly, proportional depression of the footpedal 106 is utilized for proportional control of fluid injection with the injection device, where the position of the footpedal 106 (e.g., the extent to which the footpedal 106 is depressed) corresponds to a desired flow rate of the injection device.

As discussed in more detail below, the foot controller 100 is useful as an integrated primary control foot controller when physically or wirelessly coupled to a surgical console and/or injection device. In certain embodiments, the foot controller 100 is wirelessly in direct communication with an injection device. In certain other embodiments, the foot controller 100 is physically or wirelessly coupled to a surgical console, which is in wireless communication with an injection device.

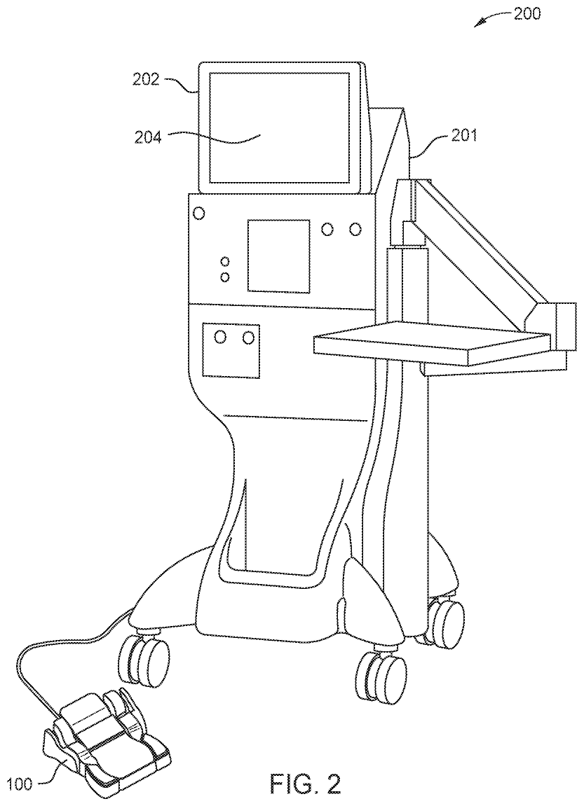

FIG. 2 illustrates a perspective view of an exemplary surgical system 200 including a surgical console 201, which is operably coupled, physically or wirelessly, to any number of user interfaces, including the foot controller 100, in accordance with certain embodiments of the present disclosure. The surgical console 201 allows a user, generally a surgeon or other medical professional, to select ophthalmic procedures and set operating parameters and modes for such processors into the surgical console 201, for example by using an electronic display screen 202 (e.g., via a touch-screen interface, mouse, trackball, keyboard, etc.), which displays a graphical user interface (GUI) 204. The electronic display screen 202 allows the user to access various menus and screens related to the functions and operations of the surgical console 201. For example, the surgeon may select a fluid delivery operation during which a handheld injection device (e.g., shown in FIGS. 3 and 4) is used to deliver fluid to intraocular tissues of the patient. As described in further detail below, in certain embodiments, surgical system 200 is configured to wirelessly control the operations of the injection device based on commands received from the surgeon through the foot controller 100.

After a fluid delivery operation or mode is selected on the surgical console 201, the surgeon can control injection with the injection device by depressing the footpedal 106. In certain embodiments, control or command signals corresponding to the position (e.g., angle or displacement) of the footpedal 106 or the amount of pressure applied thereto are transmitted from the foot controller 100 to the surgical console 201 and then relayed by the surgical console 201 to the injection device to perform injection. The surgeon controls the injection flow rate of the injection device based on the position of the footpedal 106 such that the further the footpedal 106 is depressed, the faster the fluid in the injection device is dispensed. In certain embodiments, during the injection, the injection device wirelessly communicates with the surgical console 201 and provides injection information (e.g., flow rate, fluid volume remaining or dispensed) in graphics or text to display on a display screen for the surgeon, such as electronic display screen 202 of the surgical console 201. In certain embodiments, the injection information is provided to and displayed on a display device separate from the surgical console 201, such as a display device of a high-definition visualization system. For example, the injection information is displayed on a three-dimensional (3D) organic light-emitting diode (OLED) display screen of a stereoscopic microscope workstation, which may be observed by the user through passive, polarized 3D glasses.

In certain embodiments, control or command signals from the foot controller 100 are directly transmitted to the handheld injection device to perform injection. In other words, in such embodiments, the control signals do not pass through the surgical console 201.

FIG. 3 illustrates a cross-sectional side view of a handheld injection device 300. The injection device 300 may wirelessly communicate with and receive commands from the foot controller 100 and/or surgical system 200, in accordance with certain embodiments of the present disclosure. For example, the injection device 300 is wirelessly coupled to the foot controller 100 and/or surgical system 200 to enable remote injection control, such as by operation of the foot controller 100, thus reducing or eliminating the uneven forces and tremors from the user's hand during the injection. Note that injection device 300 may be controlled by any other type of user interfaces. For example, the surgeon may trigger injection, select and change the injection flow rate, and generally operate the injection device 300 in other similar ways by communicating with the surgical console 201 through a graphical user interface 204 or other user interfaces (e.g. voice commands, other user interface devices, etc.).

The injection device 300 includes a handpiece 302, an electro-pneumatic drive unit 340, and a syringe or similar device 312 attached to the handpiece 302 and operably coupled to the drive unit 340. The injection device 300 is an automatic injection device with the drive unit 340 providing force or power to deliver an injection fluid 322 contained within the syringe 312. The injection fluid 322 may include one or more agents or materials (e.g., therapeutic agents or materials) to be delivered to intraocular tissues of a patient, for example, in solution or suspension form.

The handpiece 302 houses the drive unit 340 and the syringe 312 and may include one or more divided interior compartments therein. A distal end 304 of the handpiece 302 includes a port 306 to receive and engage the syringe 312 while a proximal end 308 of the handpiece 302 is enclosed by a removable cap 310, thus enabling access to the drive unit 340 if desired. Note that, as described herein, a distal end or portion of a component refers to the end or the portion that is closer to a patient's body during use thereof. On the other hand, a proximal end or portion of the component refers to the end or the portion that is distanced further away from the patient's body. The handpiece 302 may be formed as a single, integral component, or from multiple separate components permanently or removably coupled together. The handpiece 302 is formed of any suitable material, and is formed by any method, such as for example, injection molding or machining. In certain embodiments, the handpiece 302 is formed of a thermoplastic or metal and may be textured or contoured for improved gripping thereof by the user.

The syringe 312 includes a syringe barrel 314 having a cavity 320 at least partially defining a volume (e.g., reservoir) for injection fluid 322. A proximal end 324 of the syringe barrel 314 is open to slidably receive a stopper 334 coupled to a distal end of a plunger rod 332. In certain embodiments, the plunger rod 332 and stopper 334 may together be referred to as a plunger 333. In certain embodiments, the stopper 334 is a component of the syringe 312 and only engages with the plunger rod 332 upon insertion of the syringe 312 into the handpiece 302. A needle 328 extends from a distal end of the syringe barrel 314 for piercing of ocular tissues and delivery of the injection fluid 322 when the plunger 333 is linearly actuated. In certain embodiments, the syringe 312 is a pre-filled syringe having a predetermined volume of injection fluid 322 that is engaged with the handpiece 302 after filling. In certain other embodiments, the syringe 312 is filled after engagement with the handpiece 302. For example, the syringe 312 may be filled with injection fluid 322 by injection through a port or septum disposed through the handpiece 302. The syringe 312 may be removably or integrally attached to the handpiece 302 by any suitable mechanism. In certain embodiments, one or more mating features 330 such as flanges, grooves, or threads are formed on an outer surface of the syringe 312 to engage with and secure the syringe 312 to the handpiece 302. Similar to the handpiece 302, the syringe 312 is formed of any suitable material, and is formed by any method, such as for example, injection molding or machining.

The plunger rod 332 extends through an intermediate compartment 336 of the handpiece 302 and engages the stopper 334 at a distal end thereof. Linear movement of the plunger rod 332 through the intermediate compartment 336 causes linear actuation of the stopper 334 through the cavity 320 to direct the injection fluid 322 through the needle 328. For example, forward movement (e.g., from a proximal position to a distal position) of the plunger rod 332 forces the stopper 334 to distally move through the cavity 320 and push injection fluid 322 therefrom. In certain embodiments, the stopper 334 is formed of a suitable elastomeric material that enables slidable engagement of the stopper 334 with an interior surface of the cavity 320 while forming a fluid-tight seal. In certain other embodiments, the stopper 334 includes one or more seals to establish a fluid-tight seal for the cavity 320.

In embodiments where the drive unit 340 is an electro-pneumatic drive unit utilizing pressurized gas, such as in FIG. 3, the plunger 333 includes a flange 338 disposed at a proximal end of the plunger rod 332 that forms an interface between the plunger 333 and the drive unit 340. The flange 338 acts as a seal or plug upon which gas pressure may apply a force to cause actuation thereof. Accordingly, the flange 338 is slidably engaged with an interior surface of the intermediate compartment 336 and forms a fluid-tight seal therein. The flange 338 is therefore formed of a suitable elastomeric material or includes one or more seals at a perimeter thereof.

The drive unit 340 generally includes an actuator 342, wireless communication module 344, and a battery 346 to supply power to the actuator 342 and wireless communication module 344. The electro-pneumatic drive unit 340 depicted in FIG. 3 further includes a valve 348 and gas canister 350 containing a pressurized fluid. Examples of suitable pressurized fluids include but are not limited to carbon dioxide, nitrogen, and argon. The gas canister 350 removably couples to a proximal end of the handpiece 302 below the cap 310 by any suitable coupling mechanism or feature, such as for example, matching threads. Upon securing the gas canister 350 to the handpiece 302, pressurized fluid within the gas canister 350 is released (e.g., by puncturing a seal of the gas canister 350) into a septum 352, which is sealed by the valve 348.

The valve 348 is opened and closed by the actuator 342 to control the flow rate of the pressurized fluid through the septum 352 and into a pressurization pocket 354 on a proximal side of the flange 338. In a closed state, the valve 348 prevents any flow of fluid into the pressurization pocket 354. When the valve 348 is opened, the pressurized fluid is allowed to flow into the pressurization pocket 354 at a controlled flow rate depending on the position of the valve 348. As described above, the accumulation of pressurized gas in the pressurization pocket 354 applies a force to the proximal side of the flange 338, thereby causing forward (e.g., distal) movement of the plunger 333 to dispense the injection fluid 322 from the syringe 312. The valve 348 includes any suitable type of flow control valve operated by an electromechanical, electromagnetic or electro-pneumatic actuator 342. Suitable valves include, but are not limited to, solenoid-type valves, proportional valves, plug valves, piston valves, knife valves, or the like.

The actuator 342 is operably coupled to the wireless communication module 344 which includes wireless transmitter and receiver circuitry to relay signals (e.g., instructions) to and from the injection device 300. In particular, the wireless communication module 344 is directly or indirectly in wireless communication with the foot controller 100 to enable remote control of the injection device 300 with the foot controller 100. In certain embodiments, the wireless communication module 344 is indirectly in communication with the foot controller 100 via the surgical console 201, which may relay control signals from the foot controller 100 to the wireless communication module 344. In certain other embodiments, the wireless communication module 344 is directly in communication with the foot controller 100, thus receiving control signals directly therefrom. Upon receiving a signal from foot controller 100 or surgical console 201, wireless communication module 344 transmits a signal to actuator 342 to open or close valve 348. In certain embodiments, one or more interfaces may be used between wireless communication module 344 and actuator 342 (e.g., a digital to analogue converter, a driver circuit, etc.).

In operation, the user activates and controls actuation of the actuator 342 by operation of the foot controller 100, thus controlling the position of the valve 348 and the flow rate of pressurized gas through the septum 352. For example, the user may depress the footpedal 106 to open the valve 348 and increase the flow rate of the pressurized gas into the pressurization pocket 354, thereby increasing the force applied to the flange 338 and causing forward movement thereof. Alternatively, reducing depression of the footpedal 106 (e.g., raising a user's foot or pressing down on the footpedal 106 with the user's heel) may decrease the flow rate of the pressurized gas into the pressurization pocket 354, thereby slowing the movement of the flange 338. Applying no pressure to the footpedal 106 causes the footpedal 106 to transition into a fully undepressed state and, thereby, completely stop the flow of pressurized gas through the septum 352 altogether, and in turn, stop movement of the plunger 333. In certain embodiments, the flow rate of the pressurized gas into the pressurization pocket 354 may linearly correspond to the position of the footpedal 106. Accordingly, the injection flow rate of the injection device 300 may linearly correspond to the position of the footpedal 106. For example, a fully depressed state of the footpedal 106 corresponds with a maximum injection flow rate, while the fully undepressed state of the footpedal 106 corresponds with no injection flow.

In certain embodiments, information about the injection (e.g., flow rate and fluid volume dispensed or remaining) may be transmitted from the wireless communication module 344 to the surgical console 201 and displayed on the electronic display screen 202 while a user is performing the injection. In certain embodiments, information about the injection may be wirelessly transmitted from the wireless communication module 344 and/or surgical console 201 to a digital 2D or 3D surgical viewing system or display panel, or a 3D headset.

FIG. 4 illustrates a cross-sectional side view of an alternative injection device 400 including an electromechanical drive unit 440. Similar to the injection device 300, the injection device 400 may be configured to wirelessly communicate with and receive commands from the foot controller 100 and/or surgical system 200, in accordance with certain embodiments of this disclosure. For example, the injection device 400 is wirelessly coupled to the foot controller 100 and/or surgical system 200 to enable remote injection control, thus reducing or eliminating the uneven forces and tremors from the user's hand during the injection. Note that injection device 400 may be controlled by any other type of user interfaces. For example, the surgeon may trigger injection, select and change the injection flow rate, and generally operate the injection device 400 in other similar ways by communicating with the surgical console 201 through a graphical user interface 204 or other user interfaces (e.g. voice commands, other user interface devices, etc.).

The drive unit 440 includes an actuator 442, wireless communication module 344, and a battery 346 to supply power to the actuator 442 and wireless communication module 344. The drive unit 440 is an electromechanical drive unit and thus, utilizes electrical input to the actuator 442 to create mechanical force on a plunger 433 having a flange 438, plunger rod 432, and stopper 434. The actuator 442, such as a rotary actuator, is mechanically engaged with an elongated drive device 456 which translates movement of the actuator 442, such as rotational movement, into linear movement of the plunger 433. The actuator 442 is further in communication with the wireless communication module 344. In certain embodiments, one or more interfaces may be used between wireless communication module 344 and the actuator 442 (e.g., a digital to analogue converter, a driver circuit, etc.). Upon receiving signals from the foot controller 100 or surgical console 201, the wireless communication module 344 transmits a signal to the actuator 442 to actuate the elongated drive device 456. The elongated drive device 456 may be any suitable type of drive device, including but not limited to a drive screw, a rack engaged with a pinion, or the like. In FIG. 4, the elongated drive device 456 is depicted as a drive screw mated with the actuator 442 and the flange 438. As shown, the flange 438 forms an interface between the plunger 433 and the drive unit 440.

In operation, the user may activate and control the actuator 442 by operation of the foot controller 100, thus controlling movement of the elongated drive device 456. For example, the user may depress the footpedal 106 to rotate or linearly actuate the elongated drive device 456 in an injection direction and cause forward (e.g., distal) movement of the plunger 433, thereby forcing the injection fluid 322 out of the syringe 312. Alternatively, reducing depression of the footpedal 106 may slow the movement of elongated drive device 456 in the injection direction, thereby slowing movement of the plunger 433. Applying no pressure to the footpedal 106 causes the footpedal 106 to transition into a fully undepressed state and, thereby, completely stop the movement of the elongated drive device 456 altogether, and in turn, stop movement of the plunger 433. In certain embodiments, the movement speed of the elongated drive device 456 may linearly correspond to the position of the footpedal 106. Accordingly, the injection flow rate of the injection device 400 may linearly correspond to the position of the footpedal 106. For example, a fully depressed state of the footpedal 106 corresponds with a maximum injection flow rate, while the fully undepressed state of the footpedal 106 corresponds with no injection flow.

In certain embodiments, the user may also control the plunger 433 to move in a reverse (e.g., proximal) direction, thus enabling the injection device 400 to draw up fluid into the syringe 312 for loading (e.g., filling) thereof. For example, the user may depress a switch on the foot controller 100 to activate a reverse mode of the injection device 400, wherein subsequent depression of the footpedal 106 actuates the elongated drive device 456 in a direction opposite the injection direction. The reverse mode may include the same mechanics as described above, wherein the reverse movement speed of the elongated drive device 456 linearly corresponds to the position of the footpedal 106.

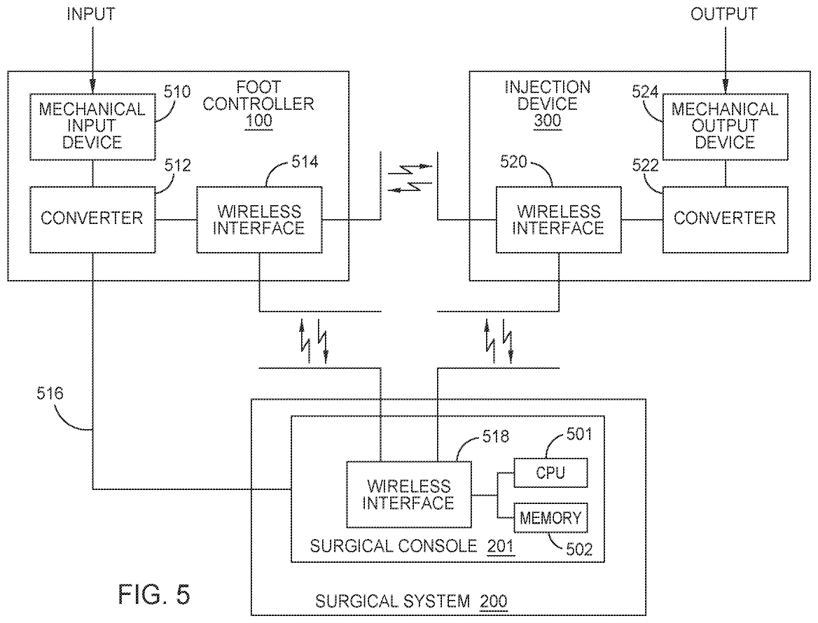

FIG. 5 illustrates an exemplary diagram showing how various components of an injection device 500 (e.g., injection devices 300, 400), surgical system 200, and foot controller 100 communicate and operate together. Foot controller 100 contains a mechanical input device 510, such as footpedal 106, which receives a mechanical input from a user and provides a control signal to signal converter 512. The control signal may include a measurement of the mechanical input device 510's position (e.g., in terms of angle or displacement), which is converted into a digital signal for relaying to surgical system 200 and/or injection device 500. Where the foot controller 100 is a wireless device, the digital signal is wirelessly relayed to surgical system 200 and/or directly to the injection device 500 via wireless interface 514. Where the foot controller 100 is wired, the digital signal is relayed to surgical system 200 via interconnect 516 and then wirelessly relayed to injection device 500 via wireless interface 518 of the surgical console 201.

The surgical console 201 includes a processor or central processing unit (CPU) 501, memory 502, and support circuits. CPU 501 may retrieve and execute programming instructions stored in the memory 502. Similarly, CPU 501 may retrieve and store application data residing in memory 502. CPU 501 can represent a single CPU, multiple CPUs, a single CPU having multiple processing cores, and the like.

Memory 502 may be one or more of a readily available memory, such as random access memory (RAM), read only memory (ROM), floppy disk, hard disk, solid state, flash memory, magnetic memory, or any other form of digital storage, local or remote. In certain embodiments, memory 502 includes instructions, which when executed by the CPU 501, performs an operation for controlling fluid delivery, as described in the embodiments herein. For example, memory 502 includes instructions that determine that the user selected an injection mode, thereby the instructions instruct the CPU 501, when executed, to activate the foot controller 100 or allow the foot controller 100 to receive commands (e.g., input) from the user. Memory 502 also has instructions that, when executed by the CPU 501, cause the surgical console 201 to control the flow rate and other operations of the injection device 500 based on the input received form the foot controller 100 (e.g., input corresponding to the position of the footpedal 106 or amount of pressure applied thereto).

As depicted in FIG. 5, wireless communication pathways are operably established between the injection device 500 and foot controller 100 and/or surgical system 200 via wireless interface 520 (e.g., wireless communication module 344). Specifically, wireless interface 520 communicatively couples to the wireless interface 514 of the foot controller 100 and/or wireless interface 518 of the surgical console 201. Each wireless interface may be implemented, for example, using low-power wireless transmitter and receiver circuitry. Thus, the control signal provided by the mechanical input device 510 is able to be converted into a digital signal and ultimately communicated to injection device 500 via wireless pathways. Upon receipt of the digital signal by wireless interface 520, the digital signal is converted by the signal converter 522 to a control signal and relayed to the mechanical output device 524, such as actuator 342 or 442, to control fluid injection parameters, such as flow rate, by the injection device 500.

In summary, embodiments of the present disclosure include structures and mechanisms for improved intraocular fluid delivery, and in particular, improved handheld injection devices for delivering therapeutic agents to intraocular tissues. The injection devices described above include embodiments wherein a user, such as a surgeon, may wirelessly control operation of the injection device via operation of a remote foot controller. The utilization of wireless remote injection control reduces or eliminates uneven application of injection force and hand tremor caused by hand-triggered devices, thus enabling precise position and flow rate control and reducing the risk of tissue damage. Accordingly, the aforementioned injection devices are particularly beneficial during injections of thin and delicate ocular tissues, such as the sub-retinal space.

While the foregoing is directed to embodiments of the present disclosure, other and further embodiments of the disclosure may be devised without departing from the basic scope thereof, and the scope thereof is determined by the claims that follow.

What is claimed is:

1. A handheld fluid injection device, comprising:
a handpiece comprising an interior compartment and a port at a distal end thereof, the port configured to receive and engage a syringe;
a plunger movably disposed within the interior compartment, a distal end of the plunger configured to slidably engage with a cavity of the syringe; and
a drive unit operatively coupled to the plunger, the drive unit comprising a wireless communication module that is in wireless communication with an input device, wherein the drive unit controls operations of the plunger based on wireless communications received from the input device for injecting fluids from the syringe.

2. The handheld fluid injection device of claim 1, further comprising:
the syringe engaged with the handpiece and comprising a cavity partially defining a reservoir for fluid.

3. The handheld fluid injection device of claim 1, wherein the input device comprises one of:
a surgical console in communication with a foot controller having a footpedal; or
a foot controller having a footpedal.

4. The handheld fluid injection device of claim 3, wherein the drive unit is controlled by operation of the foot controller.

5. The handheld fluid injection device of claim 3, wherein depression of the footpedal causes actuation of the plunger to inject fluid from the syringe.

6. The handheld fluid injection device of claim 5, wherein an injection flow rate of the handheld fluid injection device linearly corresponds to a position of the footpedal.

7. The handheld fluid injection device of claim 3, wherein a speed of movement of the plunger within the interior compartment linearly corresponds to a position of the footpedal.

8. The handheld fluid injection device of claim 1, wherein the drive unit is electro-pneumatically driven.

9. The handheld fluid injection device of claim 8, wherein the drive unit comprises a pressurized gas canister and a flow control valve controlled by an electrically-driven actuator.

10. The handheld fluid injection device of claim 9, wherein opening of the flow control valve causes pressurized gas from the pressurized gas canister to flow into the interior compartment and exert a force on the plunger.

11. The handheld fluid injection device of claim 1, wherein the drive unit is electromechanically driven.

12. The handheld fluid injection device of claim 11, wherein the drive unit further comprises an electrically-driven actuator operatively coupled to an elongated drive device that is mechanically engaged with the plunger.

13. The handheld fluid injection device of claim 12, wherein rotational movement of the actuator causes linear movement of the plunger.

14. The handheld fluid injection device of claim 3, wherein information about fluid injection is displayed on a display screen of the surgical console.

15. The handheld fluid injection device of claim 2, wherein information about fluid injection is displayed on a display screen of a visualization system.
